(19) United States
(12) Patent Application Publication
(10) Pub. No.: US 2014/0271931 A1
(43) Pub. Date: Sep. 18, 2014

(54) Anti-Acne Topical Films

(71) Applicant: Perosphere Inc., Danbury, CT (US)

(72) Inventors: Edith Mathiowitz, Brookline, MA (US); Bryan E. Laulicht, Danbury, CT (US); Sasha H. Bakhru, Providence, RI (US); Solomon S. Steiner, Mount Kisco, NY (US)

(73) Assignee: Perosphere Inc., Danbury, CT (US)

(21) Appl. No.: 14/206,523

(22) Filed: Mar. 12, 2014

Related U.S. Application Data
(60) Provisional application No. 61/777,786, filed on Mar. 12, 2013.

(51) Int. Cl. A61K 9/70; A61K 47/32; A61K 45/06; A61K 31/4402

(52) U.S. Cl. CPC A61K 9/7015; A61K 31/4402; A61K 47/32; A61K 45/06
USPC 424/713; 514/354; 514/772.6

(57) ABSTRACT

Topical formulations containing one or more pharmaceutically acceptable bioadhesive film-forming agent, one or more anti-acne agent, and an aqueous solvent in the form of a solution or suspension are described herein. The formulation may further contain one or more excipients, including evaporation suppressants, humectants, or plasticizers. When the formulation is contacted with the skin of a patient, the solvent evaporates and forms a thin, transparent, and solid bioadhesive film. The bioadhesive film adheres to the skin surface for a prolonged period of time and the anti-acne agent is released into the skin over a prolonged period of time. Typically, the bioadhesive film adheres to the skin for at least 60 minutes following administration of the formulation, preferably for at least 8 hours following administration, more preferably up to 24 hours following administration. The prolonged retention of the anti-acne agent at the site increases the amount of uptake into the skin.

### CROSS-REFERENCE TO RELATED APPLICATION

This application claims priority to U.S. Ser. No. 61/777,786, entitled “Anti-Acne Topical Films”, to Solomon Steiner, Edith Mathiowitz, Bryan Laulicht, and Sasha Bakhru, filed Mar. 12, 2013. The disclosure of which is incorporated herein by reference.

### FIELD OF THE INVENTION

The invention is in the field of formulations for treating acne, specifically, bioadhesive films containing an anti-acne agent that adhere to the skin for long periods of time providing sustained relief of the anti-acne agent.

### BACKGROUND OF THE INVENTION

Acne is a common human skin disease that afflicts the majority of all teenagers, along with a significant number of men and women of adult age. Acne vulgaris can occur anywhere on the body, most often on oily areas of the skin having high sebaceous gland concentration. These areas include the face, ears, behind the ears, chest, back, and occasionally the neck and upper arms. One causative factor for acne is increased activity of the sebaceous glands and the epithelial tissue lining the infundibulum, in which bacterial invasions cause inflamed and infected sacs to appear. Among the bacteria flora present are anaerobic, Gram positive organisms called Proprionibacterium acnes.

The increased activity of the sebaceous glands produces more sebum which consists of free and esterified fatty acids as well as unsaponifiable lipid components which results in increased skin oiliness. In inflammatory acne, the initial inflammation of hair follicle walls results from the presence of free fatty acids derived from the sebum. In the presence of bacterial lipolytic enzymes, triglycerides of the sebum are split, releasing the fatty acids. Ideally, topical formulations for the treatment of acne should be compounded with very little or no oil in the formulation and should not leave any oil film on the skin to compound the condition. However, most anti-acne agents are insoluble in water, and thus difficult to incorporate into aqueous systems. As a result, many current topical acne formulations have an oil-based vehicle, typically in the form of lotions or creams. Therefore, there is a need for aqueous anti-acne topical treatments such that undesirable side effects from increased skin oiliness are prevented.

Another major disadvantage of current anti-acne formulations, in the form of lotions and creams, is that treatment needs to be applied multiple times a day during the treatment period. This is inconvenient and can adversely affect patient compliance. The need for multiple daily applications is also not cost effective, particularly in under-developed nations. There is a need for anti-acne topical treatments that provide increased residence time at the site of the infection such that the therapeutic outcome of the anti-acne agent is enhanced.

A film-forming bioadhesion is one solution to the problem of inadequate residence time for anti-acne formulations. Venkatraman, et al., Biomaterials, 19, 1119-1136 (1998) is a review of several bioadhesive drug delivery systems. Acrylics, polyisobutylenes, and silicones were noted as viable pressure sensitive adhesives for skin applications. For sufficient and prolonged bioadhesion to occur, an intimate contact must exist between the bioadhesive and the receptor tissue, non-covalent or chemical bonds must form, and the bioadhesive must be flexible and mechanically stable.

Several bioadhesive formulations have been described as a means of topical anti-acne drug delivery. U.S. Pat. No. 6,251,371 to Holmen, et al. discloses topical formulations containing dichlorobenzyl alcohol, an anti-inflammatory compound, and a bioadhesive in the form of a gel, ointment, cream, or solution. Holmen, et al. does not disclose formation of a film.

Gels, creams, and ointments can however be irritating, messy, or sometimes visible on the skin, which may discourage patient use. Furthermore, as noted earlier, these delivery vehicles must be applied several times per day which can be a hassle and quite expensive. Repeat applications are inconvenient, and it's easy for a dose to be missed. Gels are usually formed from low molecular weight polymers and therefore does not allow high loading of the anti-acne drugs.

U.S. Pat. No. 5,658,956 to Martin, et al. discloses anti-acne formulations containing film-forming bioadhesives. Further, Martin discloses that the bioadhesives generally comprise a mucoadhesive hydrogel such as a polyacrylic acid cross linked by a polyhydroxy compound such as a carbohydrate (sugar, cyclitols) to form a substantially water-insoluble hydrogel. Martin also discloses that the formulations contains pyruvate, an antioxidant, and a mixture of saturates and unsaturated fatty acids. It is undesirable for anti-acne formulations to be oil-based due to build-up of oil on the skin.

U.S. Pat. No. 7,645,803 to Tamarkin, et al. describes a foamable composition containing a saccharide, an anti acne active agent, and at least one polymeric agent selected from a bio-adhesive agent, a gelling agent, a film-forming agent, and a phase change agent.

U.S. Pat. No. 6,280,764 to Fotinos describes a patch for topical application of an anti-acne formulation containing a backing film, a release layer and at least one adhesive polymeric matrix layer located between the backing film and the release layer. The use of a patch for delivery of anti-acne agents has limitations. Since the patch is a finite size and shape, the application area is determined by the patch and not by the dimensions of the affected site. Furthermore, applying the patch to the face is cosmetically undesirable. The patch may also make the patient warmer, and thus be a burden in hot environments. A further limitation of the patch is that percutaneous penetration of the drug is often poor.

Film formulations possess several advantages: they conform to contours (due to their elastic and flexible nature), they have increased peelability and mechanical strength, and are transparent. Furthermore, compared with transdermal patches, bioadhesive films represent an improvement because they offer more dosage flexibility and ease of use, less irritation potential, better cosmetic appearance, higher simplicity of manufacture, and longer duration of drug release. There is a need for bioadhesive formulations with sufficient and prolonged bioadhesion for increasing the absorption of anti-acne agents from polymeric drug delivery vehicles.

Therefore, it is an object of the invention to provide topical anti-acne formulations that provides increased residence time at the site of administration, and methods of making and using thereof.

It is another object of the invention to develop a formulation that is water based such that undesirable side effects from increased skin oiliness are prevented.

It is another object of the invention to provide topical anti-acne formulations that are flexible, mechanically stable, transparent, and peelable.

### SUMMARY OF THE INVENTION

The formulations described herein are topical preparations containing one or more pharmaceutically acceptable bioadhesive film-forming agents, one or more anti-acne agents, and an aqueous solvent. In one embodiment, the formulation contains one or more pharmaceutically acceptable bioadhesive film-forming agents, picolinic acid and/or picolinic acid derivatives, and an aqueous solvent. In another embodiment, the formulation contains uncrosslinked polyacrylic acid polymer as the bioadhesive film-forming agent, one or more anti-acne agents, and an aqueous solvent. The formulation may further contain one or more excipients, including, but not limited to, evaporation suppressants, humectants, and plasticizers.

When the formulation is contacted with the skin of a patient, the solvent evaporates and forms a thin, transparent, solid bioadhesive film containing solid particles of active anti-acne agents. The bioadhesive film adheres to the skin surface for a prolonged period of time. Typically, the bioadhesive film adheres to the skin for at least 60 minutes following administration of the formulation, preferably for at least 2, 3, 4, 6, 8, 10, 12, 16, 18, 20, or 24 hours following administration, most preferably up to 48 hours following administration. The prolonged retention at the site increases the amount of anti-acne agent uptake into the skin and minimizes the number of applications necessary, which should improve patient compliance and decrease the cost of treatment. The bioadhesive polymers are soluble in water and will come off on demand, for example, by washing with water.

In one embodiment, the anti-acne agent is picolinic acid or a derivative thereof. Picolinic acid is a naturally occurring, biological compound, which inhibits the growth of numerous cultured normal and transformed mammalian cells. Picolinic acid, C₆H₅NO₂, molecular weight: 123.11 g/mol, also known as 2-pyridine carboxylic acid and alpha-pyridine carboxylic acid, is readily soluble in water.

In some embodiments, the anti-acne agent has the following structure:

[chemical structure]

or a pharmaceutically acceptable salt thereof.

Wherein R₁, R₂, R₃, R₄, is selected from the group consisting of a peptide of sixteen amino acids or more with either basic or acidic amino acids predominating, carboxyl group, alkyl group, hydrogen, and halogen. The substituted picolinic acid may have an increased molecular weight and a substantially increased half-life in the blood compared to picolinic acid. The concentration of the anti-acne agent in the formulation ranges from 1% to 99% by weight/volume of the formulation, preferably from about 1% to about 20% by weight/volume of the formulation.

The formulations contain one or more film-forming bioadhesive agents, which provide sufficient retention times of the formulation on the skin. Suitable film-forming bioadhesive agents include, but are not limited to, carboxylic acid containing polymers such as acrylic acid, methacrylic acid; copolymers of acrylic or methacrylic acid; esterified polyacrylic acid polymers, such as polyacrylic acid polymers lightly crosslinked with a polyalkenyl polyethers; methacrylate polymers; maleic acid copolymers; polyvinyl pyrrolidone; polyvinyl alcohol; poly (vinyl acetate); used alone or in combination with other suitable carriers. In the preferred embodiment, the film-forming bioadhesive material is uncrosslinked polyacrylic acid polymer. The average molecular weight can vary for a given polymer but is generally from about 5000 Daltons to 1,000,000 Daltons. Preferably, the molecular weight ranges from 20,000 Daltons to 500,000 Daltons.

High molecular weights of bioadhesive polymers allow high loading of the anti-acne drugs in the bioadhesive film. Furthermore, the high molecular weight polymers provides mechanical integrity to the bioadhesive film, such that the film adheres to the skin and does not break for at least 2, 3, 4, 6, 8, 10, 12, 16, 18, 20, or 24 hours following administration.

The concentration of the film-forming bioadhesive agent in the formulation ranges from 1% to 99% by weight/volume of the formulation, preferably, from about 1% to about 80% by weight/volume of the formulation. In one embodiment, the concentration of the film-forming bioadhesive agent in the formulation ranges from about 1% to about 20% weight/volume of the formulation.

The bioadhesive film, formed after evaporation of the solvent, must be sufficiently durable to resist erosion of the film itself, for example at the edges of certain features on the face such as the nose and mouth. Furthermore, the film must be strong enough to withstand excessive attrition by the user. Higher molecular weight bioadhesive agents provide a high tensile break strength to the bioadhesive film. Preferably, the molecular weight of the bioadhesive film-forming polymer is about 25,000 Daltons to about 100,000 Daltons.

The total thickness of the composite films can vary, for examples from nanometers to microns. In one embodiment, the composite films may have an overall thickness of greater than 30 μm. In other embodiments, the composite films may have an overall thickness of from 20 μm to 100 μm, preferably 30 μm to 50 μm. The flexibility of the films allows them to adhere to the skin surface regardless of its thickness. The bioadhesive films have enough intrinsic strength and elasticity to hold the formulation onto the skin surface for at least 2, 3, 4, 6, 8, 10, 12, 16, 18, 20, or 24 hours.

The formulation may be prepared by incorporating the film-forming bioadhesive agent, the anti-acne agent, and optionally one or more excipients in a solvent. Typically, the solvents are selected for their ability to dissolve the anti-acne agent and the film-forming bioadhesive. Suitable solvents include, but are not limited to water, cyclomethicone, benzyl alcohol, propylene glycol, polyethylene glycol, propylene carbonate, ethanol, dimethyl sulphoxide, glycerin, isopropyl alcohol, isopropyl myristate, oleic acid, and combinations thereof. The concentration of the solvent in the formulation ranges from 1% to 98% by weight/volume of the formulation, preferably from about 80% to about 97% weight/volume of the formulation.

### BRIEF DESCRIPTION OF THE DRAWINGS

FIG. 1 shows FTIR analysis of a polyacrylic acid film. Polyacrylic acid displays peaks at 2900 cm⁻¹ (C—H) and 1160 cm⁻¹ (C—OH).

FIG. 2 shows FTIR analysis of a 15% w/v % picolinic acid/10% w/v % polyacrylic acid loaded film. Polyacrylic acid displays peaks at 2900 cm⁻¹ (C—H) and 1160 cm⁻¹ (C—OH). Picolinic acid displays a peak at 1590 cm⁻¹ (C═C).

FIG. 3 is a graph of the FTIR ratio of picolinic acid (1593 cm⁻¹) to polyacrylic acid (1166 cm⁻¹) as a function of the percent picolinic acid. The ratio was measured on the side of the film facing the epidermis.

FIG. 4 is a graph of the FTIR ratio of picolinic acid (1593 cm⁻¹) to polyacrylic acid (1166 cm⁻¹) as a function of the percent picolinic acid. The ratio was measured on the outer side of the film (facing away from the epidermis).

FIG. 5 shows FTIR analysis of a 0% w/v % picolinic acid/10% w/v % polyacrylic acid loaded film after 3 hours of epidermal contact. Polyacrylic acid displays peaks at 2900 cm⁻¹ (C—H) and 1160 cm⁻¹ (C—OH). Picolinic acid displays a peak at 1590 cm⁻¹ (C═C).

FIG. 6 shows FTIR analysis of a 10% w/v % picolinic acid/10% w/v % polyacrylic acid loaded film after 3 hours of epidermal contact. Polyacrylic acid displays peaks at 2900 cm⁻¹ (C—H) and 1160 cm⁻¹ (C—OH). Picolinic acid displays a peak at 1590 cm⁻¹ (C═C).

FIG. 7 shows FTIR analysis of a 0% w/v % picolinic acid/10% w/v % polyacrylic acid loaded film after 4 hours of epidermal contact. Polyacrylic acid displays peaks at 2900 cm⁻¹ (C—H) and 1160 cm⁻¹ (C—OH). Picolinic acid displays a peak at 1590 cm⁻¹ (C═C).

FIG. 8 shows FTIR analysis of a 10% w/v % picolinic acid/10% w/v % polyacrylic acid loaded film after 4 hours of epidermal contact. Polyacrylic acid displays peaks at 2900 cm⁻¹ (C—H) and 1160 cm⁻¹ (C—OH). Picolinic acid displays a peak at 1590 cm⁻¹ (C═C).

FIG. 9 shows FTIR analysis of a 0% w/v % picolinic acid/10% w/v % polyacrylic acid loaded film after 24 hours of epidermal contact. Polyacrylic acid displays peaks at 2900 cm⁻¹ (C—H) and 1160 cm⁻¹ (C—OH). Picolinic acid displays a peak at 1590 cm⁻¹ (C═C).

FIG. 10 shows FTIR analysis of a 10% w/v % picolinic acid/10% w/v % polyacrylic acid loaded film after 24 hours of epidermal contact. Polyacrylic acid displays peaks at 2900 cm⁻¹ (C—H) and 1160 cm⁻¹ (C—OH). Picolinic acid displays a peak at 1590 cm⁻¹ (C═C).

### DETAILED DESCRIPTION OF THE INVENTION

### I. Definitions

“Transparent” as used herein refers to the optical state of a medium through which light can pass through so that an object, such as the underlying skin and/or acne, can be seen through the medium. A medium is considered transparent even if only a small fraction of light passes through the medium. Thus, a clear film and a translucent film are considered transparent.

“Solid” as used herein refers to a continuous mass of a compound or a composition. The solid has a specific shape and volume and changes its shape by force or energy, as when bent, stretched, or broken. The solid may be processed into any suitable form such as a film. The solid composition can subsequently be peeled or dissolved in a suitable diluent, such as water, in order to remove it from the site of administration. Desirable, the film is not sticky to the touch or does not leave the skin feeling stiff. In particular embodiments, the term “solid” does not encompass semi-solids, such as gels.

“Adhere” as used herein refers to the ability of one material to bind, cling, stick, or attach to another material or surface for at least 1 hour. The films described herein adhere to the skin preferably for at least 2, 3, 4, 6, 8, 10, 12, 16, 18, 20, or 24 hours following administration, most preferably up to 48 hours following administration. The films may be removed by peeling or dissolved in a suitable diluent.

“Bioadhesion” or bioadhesive” as used herein refers to the ability of a material to adhere to a biological tissue, particularly the skin, for at least 1 hour. The films described herein adhere to the skin preferably for at least 2, 3, 4, 6, 8, 10, 12, 16, 18, 20, or 24 hours following administration, most preferably up to 48 hours to the skin following administration.

“Derivatives” as used herein refers to any salt, solvate or prodrug, e.g. ester, of the compound, which upon administration to the recipient is capable of providing (directly or indirectly) a compound, or an active metabolite or residue thereof. The term also refers to any products obtainable by substituting the picolinic acid ring with different functional groups to form, for example, an azide, an acid, ester, amide, or any other products thereof. The picolinic acid derivatives can also be substituted with a substituted or unsubstituted alkyl, amino acid, aryl, heteroaryl, or cycloalkyl having 3-10 carbons. Such derivatives are recognizable to those skilled in the art, without undue experimentation.

“Analogs” as used herein refers to a chemical compound or molecule made from a parent compound or molecule by one or more chemical reactions. As such, an analog can be a compound with a structure similar to that of picolinic acid or based on a picolinic acid scaffold, but differing from it in respect to certain components or structural makeup, which may have a similar or opposite action metabolically.

“Anti-acne agent” as used herein refers to any chemical and/or biological agent (i.e. an antimicrobial peptide) that when topically administered at the site of acne, leads to a visible reduction of symptoms associated with the epithelial condition of acne vulgaris. Representative anti-acne agents include, for example, salicylic acid, sulfur, glycolic, pyruvic acid, resorcinol, N-acetylcysteine, picolinic acid, picolinic acid derivatives, picolinic acid analogs, benzoyl peroxide, and retinoids such as retinoic acid and its derivatives (e.g., cis and trans, esters).

“Robust” as used herein refers to the ability of the film to withstand deformations, such as bending, stretching, strain, displacement, etc., without breaking.

### II. Formulations

The formulations described herein are topical preparations containing one or more pharmaceutically acceptable bioadhesive film-forming agents, one or more anti-acne agents, and an aqueous solvent. The formulation may be in the form of a solution or suspension. After evaporation of the solvent, the formulation forms a flexible, transparent, solid film. The formulation does not form a foam or gel on the skin after application. As used herein, “foam” is a fine bubble structure, which does not readily collapse, containing a gas dispersed within a liquid or solid continuous phase. As used herein, a gel is a semisolid system in which a liquid is trapped within an interlocking three-dimensional network. Gels are usually formed from low molecular weight polymers that dry out over time to form a solid structure. However, due to the low molecular weight of the polymer components in the gel, the solid structure will fall apart or break. The formulations disclosed herein contain a high molecular weight polymer, preferably about 25,000 Daltons or greater. After evaporation of the solvent, the mechanical integrity of the films are such that, they do not break for at least 2, 3, 4, 6, 8, 10, 12, 16, 18, 20, or 24 hours following administration. The high molecular weight film further provides an effective delivery system for the anti-acne agents. Variations and other appropriate vehicles will be apparent to the skilled artisan and are appropriate for use in the formulations described herein. The common attribute of the various formulations is the presence of the bioadhesive film-forming agent and one or more anti-acne agents dissolved and/or dispersed in the solvent.

When the formulation is contacted with a skin surface in need of treatment, the solvent evaporates and forms a transparent, robust, solid bioadhesive film. The bioadhesive film adheres to the skin surface for a prolonged period of time. Typically, the bioadhesive film adheres to the skin for at least 60 minutes following administration of the formulation, preferably for at least 2, 3, 4, 6, 8, 10, 12, 16, 18, 20, or 24 hours following administration, most preferably up to 48 hours following administration. The prolonged retention at the site increases the amount of picolinic acid uptake into the skin.

The flexibility of the film formed after evaporation of the solvent facilitates adherence to various skin profiles. For example, the film adheres to hydrated and dehydrated skin, skin with mild and severe acne, and skin with different levels of porosities and elasticity.

A. Anti-Acne Agent

The formulations contain one or more anti-acne agents. In the preferred embodiment, the anti-acne agent is picolinic acid, a picolinic acid derivative, a picolinic acid analog, or combinations thereof.

Picolinic acid is a naturally occurring degradation product of tryptophan. Picolinic acid facilitates zinc/chromium ion absorption from the intestine because of its metal ion-chelating activity. In addition, picolinic acid inhibits the growth of numerous cultured normal and transformed mammalian cells. Picolinic acid has the chemical name 2-pyridine carboxylic acid, also known as alpha-pyridine carboxylic acid, having the chemical formula C₆H₅NO₂, molecular weight: 123.11 g/mol, and is readily soluble in water.

Computer modeling shows picolinic acid binds with DnaJ and reduces or eliminates the effect of the DnaJ blocking zinc finger proteins inactivating enzymes and hence controls the inflammatory response induced by bacteria in common acne.

Other disease states involving inflammatory responses that may be susceptible to treatment using picolinic acid include arthritis and Alzheimer's disease. Arteriosclerosis has an inflammatory and proliferative component that may be blocked by picolinic acid formulations.

WO 01/60349 to Femandez-Pol et al., which is incorporated herein by reference in its entirety, discloses substituted derivatives of picolinic acid and related compounds that disrupt the binding of zinc atoms in zinc finger proteins, zinc ring proteins or other structures heretofore unknown that depend upon the inclusion of zinc or other metal ions such as transition metal ions, for stability, packaging or replication.

In some embodiments, the anti-acne agent has the following structure:

[chemical structure]

or a pharmaceutically acceptable salt thereof.

Wherein R₁, R₂, R₃, R₄, is selected from the group consisting of a peptide of sixteen amino acids or more with either basic or acid amino acids predominating, carboxyl group, alkyl group, hydrogen, and halogen. In some embodiments, the substituted picolinic acid has an increased molecular weight and/or a substantially increased half-life in the blood compared to picolinic acid.

For example R₁, R₂, R₃, R₄ can be a methyl, ethyl, propyl, isopropyl, butyl, isobutyl, secondary butyl, tertiary butyl, pentyl, isopentyl, neopentyl or similar group. Further, substitution with halogens such as fluorine, chlorine, bromine and iodine can result in effective, locally-acting active agents. The compounds can be prepared by methods generally known to the art and include pharmacologically acceptable salts thereof.

Fusaric acid is the 5-butyl derivative of picolinic acid, that is, a compound of Formula I wherein R₃ is butyl. Fusaric acid, by virtue of its butyl group penetrates the cell interior much more easily than picolinic acid, and works at least in part as a Zn/Cu chelating agent. Other lipophilic groups can be introduced to increase penetration into the cell interior.

Other suitable anti-acne agents include, but are not limited to salicylic acid, benzoyl peroxide, sulphur, retinoic acid, azelaic acid, clindamycin, adapalene, erythromycin, sodium sulfacetamide, aluminium chloride, resorcinol, dapsone, aluminum oxide, and combinations thereof.

B. Film-Forming Bioadhesive

The formulations contain one or more film-forming bioadhesive agents, which provide sufficient retention times of the formulation on the skin. Suitable film-forming bioadhesive agents include, but are not limited to, carboxylic acid containing polymers such as acrylic acid; methacrylic acid; copolymers of acrylic or methacrylic acid; esterified polyacrylic acid polymers, such as polyacrylic acid polymers lightly crosslinked with a polyalkenyl polyethers (commercially available under the trademarks CARBOPOL®); methacrylate polymers; maleic acid copolymers; polyvinyl pyrrolidone; polyvinyl alcohol; polysaccharides such as karaya gum, tragacanth gum, xanthan gum, jaraya gum, pectin, guar gum, locust bean gum, psyllium seed gum, alginates; cellulose and cellulose derivatives such as carboxy methyl cellulose, hydroxy propyl cellulose, cellulose based co-polymers; along with other substances known for use in transdermal preparations capable of forming a solid film that can adhere to tissue, used alone or in combination with other suitable carriers. In one embodiment, cross-linked polyacrylic acids are used in very dilute concentrations, preferably less than about 1%, such that the formulations are easily applied and form a smooth film. Preferably, the film-forming bioadhesive material is uncrosslinked polyacrylic acid polymer. The carboxylic acid groups in polyacrylic acid are also useful to prevent bacterial growth due to the acidity of the environment.

The average molecular weight can vary for a given polymer but is generally from about 5000 Daltons to 1,000,000 Daltons, 10,000 Daltons to 500,000 Daltons, 20,000 Daltons to 250,000 Daltons, or 20,000 Daltons to 100,000 Daltons.

The concentration of the film-forming bioadhesive agent in the formulation ranges from 1% to 99% by weight/volume of the formulation, preferably from about 1-40% by weight/volume of the formulation, more preferably from 1-20% by weight/volume of the formulation.

The consistency of the formulation can be varied by adjusting the ratio of solvent to the one or more film-forming bioadhesive materials to achieve the desired consistency for application to a particular site. It may be desirable to prepare the composition as a less viscous composition that can be applied thinly to the affected areas, or prepare a more viscous preparation for treatment of more severe inflammations.

The formulations form a dry transparent, solid adhesive film once in contact with the skin. The formulations do not form a semisolid film “gel” or a foam after evaporation of the solvent. Gels have a high aqueous component that permits greater dissolution of drugs, and also permit faster migration of drugs through a vehicle that is essentially a liquid, compared with the films disclosed herein. The release of the anti-acne agent from the films disclosed herein is via a controlled diffusion model, both at low and high drug concentrations.

The bioadhesive film, formed after evaporation of the solvent has sufficient mechanical strength to remain adhered to the skin for extended periods of time. The film must be sufficiently durable to resist erosion of the film itself, for example at the edges of certain features on the face such as the nose and mouth. Furthermore, the film must be strong enough to withstand excessive attrition by the user. Modifications can be made to the molecular weight of the bioadhesive film-forming polymer to provide films with a high tensile break strength. In one embodiment, the molecular weight of the bioadhesive polymer is at least 25,000 Daltons, preferably about 30,000 Daltons or greater.

The total thickness of the composite films can vary from nanometers to microns. In one embodiment, the composite films may have an overall thickness of greater than 30 μm. In other embodiments, the composite films may have an overall thickness of from 20 μm to 100 μm, preferably 30 μm to 50 μm. It is known in the art that very thin films (less than 1 μm) possess higher adherence strength than thicker films. Although the films have a thickness greater than 1 μm, their flexibility allows them to stick to the skin surface. The bioadhesive films have enough intrinsic strength and elasticity to hold the formulation onto the skin surface for greater than 1, 2, 4, 6, 8, 10, 12, 16, 20, 24, 36, or 48 hours.

C. Solvent

The formulation may be prepared by incorporating the one or more film-forming bioadhesive agents, the anti-acne agents, and optionally one or more excipients in a solvent. The formulation can be in the form of a solution and/or a suspension. The type of matrix that may be formed depends on the solubilities of the anti-acne agent and the film-forming bioadhesive. All of the solutes can be soluble in the solvent thereby forming a solution. Alternatively, one or more of solutes are insoluble or partially soluble thereby forming a suspension. Typically, the solvents are selected for their ability to dissolve the anti-acne agent and the film-forming bioadhesive.

Suitable solvents include, but are not limited to water, cyclomethicone, benzyl alcohol, propylene glycol, polyethylene glycol, propylene carbonate, C1 to C4 alcohols such as ethanol, isopropyl alcohol, dimethyl sulphoxide, glycerin, isopropyl myristate, oleic acid, and combinations thereof.

The concentration of the solvent in the formulation ranges from 1% to 98% by weight/volume of the formulation, more preferably from about 80-97% by weight/volume of the formulation.

The solvent evaporates within about 5 minutes following administration of the formulation, preferably within about 3 minutes following administration. The anti-acne agent precipitates as the solvent evaporates. Faster evaporation of the solvent provides smaller particle size of the anti-acne agent in the formulation. Slow evaporation of the solvent may result in large particle size of the anti-acne agent, which are released at a slower rate into the skin.

D. Excipients

The formulation may optionally contain one or more excipients selected from the group consisting of an evaporation suppressant, humectants, plasticizing agents, and/or permeation enhancers. One of ordinary skill in the art would know how to add the excipients in an effective amount that will not compromise the mechanical and adhesive properties of the film, which is measured in all formulation by adhering to the skin for at least 1 hour.

1. Evaporation Suppressant

The length of the drying time can be controlled to minimize loss or ineffectiveness in the final film. Furthermore, the aqueous solvent is removed from the film in a manner such that the uniformity, or more specifically the non-self-aggregating uniform heterogeneity, that is obtained in the wet film is maintained. A controlled drying process may be achieved through addition of an evaporation suppressant. The evaporation suppressant functions by forming a thin film at the surface of the film which retards evaporation. This thin film formation and resultant evaporation suppressant activity of the evaporation suppressant is produced by the evaporation and surface chilling which occur when the aqueous solvent are exposed to air.

High molecular weights bioadhesive polymers also act as an evaporation suppressant. The higher molecular weight polymers provide slower rates of evaporation of the solvent from the formulation, especially from the inner surface (surface in contact with the skin) of the film. Preferably, the bioadhesive polymer is polyacrylic acid with molecular weights ranging from 25,000 Daltons to 500,000 Daltons. Polyacrylic acid polymers have large numbers of carboxylic acid groups and therefore will retain moisture.

In the preferred embodiments, the solvent evaporates within 5 minutes or less following administration.

Suitable evaporation-suppressing agents include but are not limited to glycerin, polyethylene glycol, or other aliphatic alcohols and ether alcohols containing 16 to 30 carbons, such as described in U.S. Pat. No. 3,146,059.

The concentration of the evaporation suppressant in the formulation ranges from 0.5% to 10% by weight/volume of the formulation, more preferably from about 0.5-5% by weight/volume of the formulation.

2. Humectant

In one embodiment, the formulation is modified to contain one or more humectants. A humectant is a hygroscopic substance with a tendency to form hydrogen bonds with molecules of water. The humectant typically has several hydrophilic groups, such as hydroxyl groups, amines or carboxyl groups. The formulation may contain any suitable amount of the humectant to ensure that the formulation retains the necessary level of water.

Humectants retain water molecules that would otherwise evaporate from the formulation over the period when the formulation is applied to the skin. A loss of water in the formulation may cause any number of effects, among them, decreased bioavailability of the anti-acne agent, loss of interfacial contact between the film and the skin site due to flaking of the film. Any one of the above effects can interfere with the film's performance.

Suitable humectants include, but are not limited to polyhydric alcohols, such as glycerin, sorbitol, xylitol, butylene glycol, polyethylene glycol, propylene glycol, urea, propylene glycol, sodium lactate, sodium pyrrolidone carboxylic acid (PCA), hyaluronic acid (sodium salt), carrageenan and agarose.

Suitable amounts of the humectant in the hydrogel range from about 0.1% to about 10% (wt/vol), preferably the amount ranges from 0.1 to 5% (wt/vol).

3. Plasticizer

In one embodiment, the formulation is modified to contain one or more plasticizers. The plasticizers provide a flexible film, which is comfortable to the patient when placed on his/her skin. If the film has an excessive level of plasticizer, the mechanical strength of the film will be reduced. The concentration of the plasticizer is from about 1% to about 20% weight/volume of the composition, preferably from about 1% to about 10% weight/volume of the composition.

Other suitable plasticizers include, but are not limited to triacetin, dimethyl phthalate, diethyl phthalate, dibutyl phthalate, dibutyl sebacate, triethyl citrate, tributyl citrate, triethyl acetyl citrate, castor oil and acetylated monoglycerides.

The humectant may also act as a plasticizer to provide a flexible film, which is comfortable to the patient when placed on his/her skin.

4. Skin Permeation Enhancers

The formulations described herein may include at least one skin permeation enhancer. Suitable skin permeation enhancers include, but are not limited to, benzyl alcohol, linoleic acid, alpha-linolenic, oleic acid, cod-liver-oil, methanol, menthol derivatives, squalene, glycerol derivatives, and sodium taurocholate.

Suitable skin permeation enhancers are well known to one of skill in the art and are also described in U.S. Pat. No. 5,947,921 to Johnson, et al. Skin permeation enhancers are generally discussed below.

### III. Method of Treatment

The formulations described herein deliver an effective amount of one or more agents to treat acne. The formulation can be applied wherever the patient has superficial skin lesions or infections, such as the face and/or back. The adherence profile of the bioadhesive film to the skin surface may be affected by one or more factors, including, the molecular weight, concentration, flexibility, and the spatial conformation of the ingredients in the formulation.

The film adheres to the skin surface by forces that are measurable and by any number of mechanisms such as, but not limited to the following: hydrogen-bonding, ionic interaction, hydrophobic interaction, van der Waals interaction, or combinations thereof. The strength of adherence can be measured by standard tests for measuring the force, e.g. in dynes per square centimeter, as disclosed in U.S. Pat. No. 4,615,697 to Robinson. The composition conveniently can be removed at will, by peeling or by washing with soap and water.

After application, the solvent evaporates to leave a transparent, protective, solidified, and adherent film on the skin surface to which it has been applied. The solidified film residue contains the one or more anti-acne agent, the one or more film-forming bioadhesive, and optionally, any suitable excipient. By forming an adhesive film, the film-forming bioadhesive permit a sustained, continuous release and a prolonged exposure of the agent or agents to the skin. Continuous exposure of the skin to the medication is maintained as long as the coating stays in place. The film, therefore can effect symptomatic relief with less frequent applications.

In some embodiments, after evaporation of the solvent, the bioadhesive film adheres to the patient's skin, with a residence time of at least 60 minutes following administration. In other embodiments, the residence time of the bioadhesive film is from about 60 minutes to about 12 hours, preferably from about 60 minutes to 24 hours, more preferably from about 60 minutes to 48 hours. Preferably, the residence time of the bioadhesive film is greater than 2, 3, 4, 6, 8, 10, 12, 16, 18, 20, or 24 hours. The bioadhesive film may be removed by peeling or by complete dissolution of the film in a solvent, for example water.

Because the adherent properties of a film-forming bioadhesive allow for extended and continuous exposure of a skin inflammation to the anti-acne agent, reduced concentration formulations are possible. In some embodiment, the anti-acne agent is released into the skin for at least 2, 3, 4, 6, 8, 10, 12, 16, 18, 20, or 24 hours, preferably for up to 48 hours following administration. The amount of formulation to be used can therefore be adjusted as appropriate. A pharmaceutically active and acceptable preparation of picolinic acid or derivative in a concentration of approximately 1% to approximately 99% weight/volume, preferably 1% to 40% weight/volume, most preferably, about 1% to 20% weight/volume of the formulation. Gels uses low molecular weight polymers and do not allow high drug loading.

A wide range of quantities of the compositions can be employed to provide an anti-acne benefit. Quantities of the present compositions which are typically applied to provide an anti-acne benefit can range from about 0.1 mg/cm² to about 10 mg/cm². A particularly useful amount to use is about 2.5 mg/cm² to about 5 mg/cm².

For most acne related symptoms, one or two daily applications will be sufficient to promote regression or disappearance of the targeted skin lesions. For certain less respondent lesions, three daily applications may be required to effect disappearance of symptoms. Other dermatological disorders may require application every second day to realize symptomatic relief.

In one embodiment, the anti-acne bioadhesive films reduce inflamed lesions by greater than about 5, 10, 15, 20, 25, 30, 35, or 40% after one week, two weeks, three weeks, four weeks, five weeks, or six weeks. In one embodiment, the anti-acne bioadhesive films prevent the formation of inflamed and inflamed lesions due to acne.

A. Therapeutic Indications

The preparation may be used for local topical delivery to any location where reduction of inflammation due to acne is required or desirable. The formulation can also be used to treat a variety of conditions, including virally induced or spontaneous proliferative diseases of the skin or mucous membranes in human and animal subjects.

The preparation can be applied to skin to control warts and herpes infections and to toe nails and fingernails, for example, to treat fungal infections.

### IV. Kits

Kits containing formulations from about 1% to about 40% of the formulation weight/volume of the formulation are described herein. In one embodiment, the formulation contains about 10% by weight/volume of picolinic acid, picolinic acid derivatives, or picolinic acid analogs in the form of the free acid or a pharmaceutically acceptable salt. The kit may include a container, such as a tube dispenser or a propellant-based device, containing the formulation, for example, in the form of a solution or suspension. The kit may further contain instructions for administering the formulation as well as medical supplies for administering the formulation, such as gloves, mechanical pump, applicators, such as a brush, q-tip or swab, and combinations thereof.

### EXAMPLES

The present invention will be further understood by reference to the following non-limiting examples.

### Example 1

### Preparation of Polyacrylic Acid and Picolinic Acid Films

Several film cast samples have been evaluated for their potential use in epidermal delivery. Polyacrylic acid and picolinic acid were combined in the following ratios (Table 1) in individual 20 ml glass scintillation vials.

Next, the combined polymer and drug mixtures were dissolved in their respective Milli-Q H₂O volumes and vortexed for 15 seconds. Upon complete mixing, the samples were then placed on the bench top at room temperature overnight to allow complete dissolution. All samples produced clear solutions. Polymer concentrations of 1%, 5% and 10% w/v % were prepared and drug loading in each individual polymeric mixture ranged from 1% to 15% picolinic acid loading.

Upon complete dissolution, 0.1 ml droplets were plated out onto a labeled polyamide plastic bag utilizing a 1 ml slip tip syringe. The droplets were then allowed to dry on the bench top for 24 hours at room temperature producing film casts. Once fully dried, the films were removed and stored for further analysis making note of which side was in contact with the polyamide film surface.

Each film was stable for a period of up to 6 months at room temperature and easily peeled off of the surface for further analysis. Films with different thicknesses could also be fabricated easily by altering the concentration of the polymeric solution as well as utilizing different volumes in the film casting process.

TABLE 1
Ratio of Picolinic Acid to Polyacrylic Acid

| | | | | Polymer/ |
|---|---|---|---|---|
| Drug/Polymer (w/w %) | | | | Solvent |
| Picolinic | | Polyacrylic | | (10 w/v %) |
| Acid | Act. Weight | Acid | Act. Weight | Act. Volume |
| 0% | 0 mg ± 0 | 100% | 1000 mg ± .1 | 10.00 ml |
| 1% | 10 mg ± .1 | 99% | 990 mg ± .1 | 9.90 ml |
| 2% | 20 mg ± .1 | 98% | 980 mg ± .1 | 9.80 ml |
| 3% | 30 mg ± .1 | 97% | 970 mg ± .1 | 9.70 ml |
| 4% | 40 mg ± .1 | 96% | 960 mg ± .1 | 9.60 ml |
| 5% | 50 mg ± .1 | 95% | 950 mg ± .1 | 9.50 ml |
| 6% | 60 mg ± .1 | 94% | 940 mg ± .1 | 9.40 ml |
| 7% | 70 mg ± .1 | 93% | 930 mg ± .1 | 9.30 ml |
| 8% | 80 mg ± .1 | 92% | 920 mg ± .1 | 9.20 ml |
| 9% | 90 mg ± .1 | 91% | 910 mg ± .1 | 9.10 ml |
| 10% | 100 mg ± .1 | 90% | 900 mg ± .1 | 9.00 ml |
| 15% | 150 mg ± .1 | 85% | 850 mg ± .1 | 8.50 ml |

Fourier Transforming Infrared Spectroscopy Analysis of Polyacrylic Acid

In order to analyze the presence of picolinic acid in the polyacrylic acid composite film, Fourier Transforming Infrared Spectroscopy (FTIR) was utilized. An FTIR analysis of a polyacrylic acid film is presented in FIG. 1. This was performed with the Perkin Elmer Spectrum One FTIR (Perkin Elmer, Shelton, Conn.) using ATR.

FTIR of Picolinic Acid Loaded Polyacrylic Acid Films

In order to determine the distribution of the drug throughout the film, picolinic acid loaded samples were evaluated using FTIR ATR from both sides. The bottom side (the side that will be placed on the epidermis) may contain a different concentration of picolinic acid due to gravitational settling of the drug in the film. FTIR ATR was run using a Perkin Elmer Spectrum One FTIR (Perkin Elmer, Shelton, Conn.). It was found after analysis that select wavelengths could be chosen and used in a ratio calculation to produce a standard curve with relation to picolinic acid concentration in the film. This will be further discussed below. FIGS. 2 through 12 depict some of the samples that were run to analyze and calibrate the system.

Calibration Curve for Ratio Analysis of FTIR Data

The following graphs represent a ratio-based analysis of the data portrayed in the previous section. By choosing two wavelengths, specifically 1593 cm⁻¹ for picolinic acid and 1166 cm⁻¹ for polyacrylic acid, we can utilize their associated wavelengths for each concentration sample to establish a standard concentration curve (FIG. 13). This calculation was performed for both sides of the films by dividing the wavelength of the picolinic acid by the wavelength for the polyacrylic acid. Performing this calculation for both sides thus allows us to control for differences in drug settling in the film drying process.

### Results:

The two sides of the casted films seemed to be loaded with drug at almost the same concentration, which may indicate that there is very good distribution of the drug throughout the film.

### Example 2

### FTIR ATR and Electron Microscopy Study to Evaluate Performance of Picolinic Acid Loaded Polyacrylic Acid Films

FTIR ATR

In order to determine the ability of the films to adhere to the skin, several formulations were applied directly to a rat skin. Picolinic acid loaded samples were administered by slip tip syringe such that solid epidermal contact was achieved on a shaved rat. Upon drying of the film on the epidermis, the samples showed good mechanical strength and epidermal adherence over a 24 hour period. Once the 3, 4 and 24 hour time sequences had elapsed, the samples were found to be durable enough for peeling from the skin with note as to which side was in contact with the epidermis. Once removed, the samples were evaluated utilizing FTIR ATR and SEM on both sides. However, during data analysis, it was determined that the adherence of skin to the polymer film produced too much interference during FTIR ATR scanning of the material to produce meaningful data. FTIR ATR was run using a Perkin Elmer Spectrum One FTIR (Perkin Elmer, Shelton, Conn.).

Electron Microscopy

In order to determine the surface morphology and distribution of the drug on the surface of the film, picolinic acid loaded samples were evaluated using scanning electron microscopy (SEM) on both sides. The bottom side (epidermis side) due to the film casting process may contain a higher concentration of picolinic acid due to gravitational settling of the drug in the film. Through SEM image analysis it was determined that the films produced had a thickness up to 30 μm with drug particles visible on both sides. SEM was performed using a Hitachi S-2700 (Hitachi, Tokyo, Japan).

In order to determine the effects of epidermal contact on surface morphology and drug release from the surface of the films, picolinic acid loaded samples were once again administered by slip tip syringe such that solid epidermal contact was established. Upon drying on the epidermis, the samples were allowed to adhere for time periods ranging from 3 hours to 24 hours. Once the required time had elapsed, the samples were peeled from the epidermis with note as to which side was in contact with the skin. Now removed, the samples were evaluated using scanning electron microscopy (SEM) on both sides. The bottom side (epidermis side) was found to possess epidermal adherence determined by the presence of skin cells and hair follicles as observed in the SEM images. This adherence is thought to hinder our FTIR ATR analysis and thus reinforces the need to explore alternative routes of in vivo drug release analysis.

### Results:

SEM image analysis showed that the films produced for in vivo analysis possessed a thickness of up to 30 μm with picolinic acid particles visible on both sides. This indicates distribution of picolinic acid throughout the film.

The SEM images of the external side (not in contact with the epidermis) of the film showed that the scaffold of the film was maintained up to 24 hours. The SEM images of the internal side (in contact with the epidermis) of the film showed that quantity of picolinic acid reduces over time, which is indicative of release into the dermis of live rats.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A formulation for treating acne comprising:
one or more pharmaceutically acceptable bioadhesive film-forming agents, one or more anti-acne agents selected from the group consisting of picolinic acid, picolinic acid analogs, and picolinic acid derivatives, and an aqueous solvent,
wherein the film-forming agent is present in an amount from 1-99% weight/volume of the formulation,
wherein the anti-acne agent is present in an amount from 1-99% weight/volume of the formulation;
wherein the formulation, upon application to a skin surface, forms a transparent, solid film after evaporation of the solvent; and
wherein the film adheres to the skin over a period of time greater than about 1 hour.

2. A formulation for treating acne comprising:
one or more anti-acne agents, an uncrosslinked polyacrylic acid bioadhesive film-forming agent, and an aqueous solvent,
wherein the film-forming agent is present in an amount from 1-99% weight/volume of the formulation, and
wherein the anti-acne agent is present in an amount from 1-99% weight/volume of the formulation;
wherein the formulation, upon application to a skin surface, forms a transparent, solid film after evaporation of the solvent; and
wherein the film adheres to the skin over a period of time greater than about 1 hour.

3. The formulation of claim 1, wherein the film-forming agent is present in an amount ranging from 1% to 20% weight/volume of the formulation.

4. The formulation of claim 1, wherein the anti-acne agent is present in an amount ranging from 1% to 20% weight/volume of the formulation.

5. The formulation of claim 1, further comprising one or more excipients selected from the group consisting of evaporation suppressants, humectants, plasticizing agents, and permeation enhancers.

6. The formulation of claim 5, wherein the evaporation suppressant is selected from the group consisting of glycerin, and polyethylene glycol, and other aliphatic alcohols and ether alcohols containing 16 to 30 carbons.

7. The formulation of claim 6, wherein the evaporation suppressant is present in an amount ranging from 1% to about 10% weight/volume of the formulation.

8. The formulation of claim 5, wherein the humectant is selected from the group consisting of glycerin, sorbitol, xylitol, butylene glycol, polyethylene glycol, propylene glycol, urea, sodium lactate, sodium pyrrolidone carboxylic acid (PCA), hyaluronic acid, carrageenan, and agarose.

9. The formulation of claim 8, wherein the humectant is present in an amount ranging from 1% to about 10% weight/volume of the formulation.

10. The formulation of claim 5, wherein the plasticizing agent is selected from the group consisting of propylene glycol, triacetin, dimethyl phthalate, diethyl phthalate, dibutyl phthalate, dibutyl sebacate, triethyl citrate, tributyl citrate, triethyl acetyl citrate, castor oil, and acetylated monoglycerides.

11. The formulation of claim 10, wherein the plasticizing agent is present in an amount ranging from 1% to about 20% weight/volume of the formulation.

12. The formulation of claim 1, wherein the aqueous solvent is selected from the group consisting of water, cyclomethicone, benzyl alcohol, propylene glycol, polyethylene glycol, propylene carbonate, ethanol, dimethyl sulphoxide, glycerin, isopropyl alcohol, isopropyl myristate, oleic acid, and combinations thereof.

13. The formulation of claim 12, wherein the solvent is present in an amount ranging from 1 to 98% weight/volume of the formulation.

14. The formulation of claim 1, wherein the solvent is selected from the group consisting of ethanol and isopropyl alcohol.

15. The formulation of claim 1, wherein the average molecular weight of the bioadhesive film forming agent is about 5,000 Daltons to 1,000,000 Daltons, preferably 10,000 Daltons to 100,000 Daltons, more preferably 10,000 Daltons to 75,000 Daltons.

16. The formulation of claim 15, wherein the film-forming agent is selected from the group consisting of polyacrylic acid, uncrosslinked polyacrylic acid, methacrylic acid; copolymers of methacrylic acid; esterified polyacrylic acid polymers; maleic acid copolymers; polysaccharides; polyvinyl pyrrolidone; polyvinyl alcohol; acrylic polymers; acrylic copolymers; methacrylate polymers; methacrylate copolymers; cellulose based polymers; cellulose based co-polymers; and combinations thereof.

17. The formulation of claim 16, wherein the film-forming agent is uncrosslinked polyacrylic acid.

18. The formulation of claim 17, wherein the anti-acne agent is picolinic acid.

19. The formulation of claim 2, wherein the anti-acne agent is selected from the group consisting of salicylic acid, sulfur, glycolic, pyruvic acid, resorcinol, N-acetylcysteine, picolinic acid, picolinic acid derivatives, picolinic acid analogs, benzoyl peroxide, and retinoids.

20. A method for the treating acne in a patient in need thereof, comprising administering to the patient a formulation comprising
one or more pharmaceutically acceptable bioadhesive film-forming agents, one or more anti-acne agents selected from the group consisting of picolinic acid, picolinic acid analogs, and picolinic acid derivatives, and an aqueous solvent,
wherein the film-forming agent is present in an amount from 1-99% weight/volume of the formulation,
wherein the anti-acne agent is present in an amount from 1-99% weight/volume of the formulation;
wherein the formulation, upon application to a skin surface, forms a transparent, solid film after evaporation of the solvent; and
wherein the film adheres to the skin over a period of time greater than about 1 hour.

21. The method of claim 20, wherein the bioadhesive film-forming agent is uncrosslinked polyacrylic acid.

22. The method of claim 20, wherein the anti-acne agent is released from the bioadhesive film into the skin for at least 2, 6, 10, 16, 20, 24 hours, up to 48 hours.
